(19) United States
(12) Patent Application Publication
(10) Pub. No.: US 2025/0221908 A1
(43) Pub. Date: Jul. 10, 2025

(54) NOVEL SOLID COSMETIC COMPOSITIONS FOR HYGIENE AND CARE OF THE SKIN AND HAIR

(71) Applicant: PIERRE FABRE DERMO-COSMETIQUE, LAVAUR (FR)

(72) Inventors: Emmanuelle GUILLET, CASTRES Cédex (FR); Jean-Luc BLETTERY, CASTRES Cédex (FR); Anne-Laure GAUDRY, CASTRES Cédex (FR)

(73) Assignee: PIERRE FABRE DERMO-COSMETIQUE, LAVAUR (FR)

(21) Appl. No.: 18/847,144

(22) PCT Filed: Mar. 13, 2023
(86) PCT No.: PCT/EP2023/056350

§ 371 Date: Sep. 13, 2024

(30) Foreign Application Priority Data
Mar. 14, 2022 (FR) 2202195

(51) Int. Cl. A61K 8/60; A61K 8/02; A61K 8/37; A61K 8/44; A61K 8/49; A61K 8/67; A61K 8/73; A61K 8/92; A61K 8/9789; A61K 8/9794; A61Q 5/02; A61Q 19/10

(52) U.S. Cl. CPC A61K 8/602; A61K 8/0216; A61K 8/37; A61K 8/442; A61K 8/4953; A61K 8/675; A61K 8/735; A61K 8/922; A61K 8/9789; A61K 8/9794; A61Q 5/02; A61Q 19/10; A61K 2800/30

(57) ABSTRACT

The invention relates to a solid cosmetic composition containing: a mixture of surfactants comprising at least one anionic surfactant selected from acyl glutamic acids and their salts, at least one non-ionic surfactant selected from alkyl glucosides in which the alkyl radical comprises 8 to 16 carbon atoms, and at least one anionic surfactant selected from acyl lactylic acids and their salts, and at least one cosmetically acceptable carrier. This composition is free of surfactant containing an isethionic acid or isethionate group and of surfactant containing a sulfosuccinic acid or sulfosuccinate group. It is particularly suitable for hygiene and care of the skin and hair.

### FIELD OF THE INVENTION

The present invention relates to a cosmetic composition in solid form as well as the use of such a composition for the washing and/or care of the skin and/or hair. The invention also relates to a cosmetic treatment method for hygiene and/or care of the skin and/or hair using such a composition.

### PRIOR ART

The solid cosmetics market is booming at the moment and many companies are bringing to the market compositions in solid form, which are more environmentally friendly than liquid compositions, for washing and/or caring for the face, body or hair.

The oldest solid cosmetic compositions, widely used by consumers, are soaps, which are produced by a saponification reaction. However, owing to their high pH, usually greater than 10, soaps are very harsh on the skin, in particular sensitive skin, and not suitable for use on hair.

Syndets, synthetic surfactants, have more recently emerged as an advantageous alternative to products produced by saponification. Indeed, syndets have the cleansing properties of conventional soaps whilst being less harsh on the skin. Syndets contained as foaming agents in solid cosmetic compositions currently available on the market are predominantly of the type containing an isethionate group, such as sodium cocoyl isethionate (INCI name), or a sulfosuccinate group, such as disodium lauryl sulfosuccinate (INCI name). However, such substances, which have good lathering properties, are not natural and/or COSMOS-approved, a standard which defines the criteria that have to be complied with to provide consumers with a guarantee that their products are genuine organic or natural cosmetics manufactured according to the highest possible standards of sustainability. However, consumers are increasingly watchful regarding the composition of their cosmetic products. They therefore look for products free of chemicals, preferring natural ingredients, renowned for their superior tolerance and affinity with the skin, and which are more environmentally friendly.

The extract from the Mintel database, access number 8729463, “Volume & Anti-Frizz 100% Natural Custom Solid Shampoo”, 2021, describes a shampoo in solid form devoid of surfactant containing an isethionic acid or isethionate group and of surfactant containing a sulfosuccinic acid or sulfosuccinate group, and comprising, as surfactants, substances with the INCI names sodium cocoyl glutamate, sodium lauroyl lactylate, as well as arachidyl glucoside in a mixture with arachidyl alcohol and behenyl alcohol. However, such a shampoo is very hard and sticky, making it difficult to use.

The present invention aims to overcome the drawbacks of the solid cosmetic compositions proposed in the prior art, in particular the drawbacks outlined above, by proposing a solid cosmetic composition with a physiological pH, which is gentle on the skin, contains a maximum of natural ingredients, and which in particular is devoid of any surfactant of the isethionate or sulfosuccinate type, whilst at the same time having at least as good, if not better, lathering properties than those of the prior art compositions that use these non-natural syndets. Additional objectives of the invention include for this composition to contain as few ingredients as possible, and for it to be suitable for use on all types of skin, including sensitive skin and atopic skin.

The invention also aims for this solid composition to have sufficient consistency and not be too hard so that it does not crumble when it is handled or stored over time, without however being too soft and too sticky so that it would be impractical and unpleasant to use.

### DISCLOSURE OF THE INVENTION

The present inventors discovered that these objectives are achieved by using a particular combination of natural syndets, this combination unexpectedly having particularly good lathering properties when in contact with water, and enabling the formation of solid compositions that are both not very sticky and not very hard.

According to a first aspect, the present invention proposes a solid cosmetic composition, in particular suitable for topical use, in particular on skin and hair. This composition contains:
a mixture of surfactants comprising at least one anionic surfactant selected from acyl glutamic acids and their cosmetically acceptable salts, at least one non-ionic surfactant selected from alkyl glucosides in which the alkyl radical comprises from 8 to 16 carbon atoms, and at least one anionic surfactant selected from acyl lactylic acids and their cosmetically acceptable salts,
and at least one cosmetically acceptable excipient.

In addition, this composition comprises less than 5% by weight of water relative to the total weight of the composition, and it is devoid of surfactant containing an isethionic acid or isethionate group and of surfactant containing a sulfosuccinic acid or sulfosuccinate group.

A solid composition is defined in the present description, in a conventional manner, as a composition in block form which does not flow under the effect of its weight. This definition excludes in particular powdered forms. More specifically, in accordance with the present invention, solid composition is understood to be any composition having a hardness (compression strength) greater than or equal to 20 grams at ambient temperature, i.e. at a temperature comprised between 2° and 25° C. This hardness can be measured by penetrating a matrix of the composition, to a thickness of 8 mm and at a speed of 1 mm/s, with a cylindrical probe having a diameter of 0.8 cm, and measuring the penetration force of the probe. Any texture analysis device can be used for this purpose, in particular a TA.XT+ analyser marketed by Stable Micro Systems. The solid cosmetic composition according to the invention is of the type intended to be used, in particular for skin and/or hair hygiene and/or care, directly in its solid form, as opposed to solid compositions intended to be dissolved in water before they are used so as to constitute a liquid composition, which is the final form in which they are meant to be used. The composition according to the invention therefore preferably does not contain any substances capable of creating agitation upon contact with water, such as an effervescent base, in particular a combination of an organic acid and an alkaline and/or alkaline-earth hydrogen carbonate, for example citric acid and sodium carbonate. It preferably does not contain any of the gums listed below either, which are conventionally used to stabilise aqueous phases in liquid compositions, and the presence of which is detrimental to the consistency of the composition in view of the very small quantity or even absence of water in the composition: xanthan gum, diutan gum, gellan gum, sclerotium gum, succinoglucan gum, gum arabic, locust bean gum, Tamarindus indica seed gum, Caesalpinia spinosa gum. The solid cosmetic composition according to the invention can, for example, be packaged in the form of a rectangular bar, round bar, ball or stick, optionally in a refillable packaging device, etc.

In the present description, “cosmetically acceptable” is understood to mean that the salt or excipient is suitable for use in contact with human and animal cells, in particular skin or hair cells. The excipient preferably has a pleasant smell, colour and feel, and does not generate unacceptable discomfort likely to divert a user from using the composition according to the invention.

In the present description, “at least one” is understood to mean a single compound or a mixture of several such compounds. Thus, the mixture of surfactants of the composition according to the invention contains:
a single anionic surfactant selected from acyl glutamic acids and their cosmetically acceptable salts, or a plurality of such anionic surfactants,
a single non-ionic surfactant selected from alkyl glucosides in which the alkyl radical comprises from 8 to 16 carbon atoms, or a plurality of such non-ionic surfactants,
a single anionic surfactant selected from acyl lactylic acids and their cosmetically acceptable salts, or a plurality of such anionic surfactants.

Such a specific mixture of surfactants, which are all natural, advantageously ensures good lathering properties of the composition when it comes into contact with water as well as a good quality of the lather formed. Without presuming to know the underlying phenomena of this performance of the mixture of surfactants according to the invention, it can be thought that they are due to a synergistic effect of the particular groups carried by these surfactants, in particular the glutamic acid/glutamate, glucoside and lactylic acid/lactylate groups, as well as the chain length of the alkyl radical of the non-ionic surfactant, which contributes to its lathering properties. This performance is advantageously achieved in the absence in the composition of the syndets conventionally used in solid cosmetic compositions for skin and hair hygiene or care, which have the drawback of a non-natural origin.

Thus, the composition according to the invention is devoid of any surfactant containing an isethionic acid or isethionate group, in particular any surfactant selected from acyl isethionic acids or their salts, such as the substance with the INCI name sodium cocoyl isethionate. It is also devoid of any surfactant containing a sulfosuccinic acid or sulfosuccinate group, in particular any surfactant selected from alkyl sulfosuccinic acids or their salts, such as the substance with the INCI name disodium lauryl sulfosuccinate.

In addition, the solid composition according to the invention is advantageously devoid of soap, i.e. a substance obtained by saponification, such as the substances with INCI names sodium palmate, sodium cocoate, sodium olivate, sodium stearate, etc. The pH of the composition according to the invention is thus advantageously close to the physiological pH of the skin. It is preferably comprised between 4.5 and 7, more preferably between 4.5 and 6 and particularly preferably between 5 and 6. The pH of the solid composition according to the invention can be measured by dissolving the composition in water, for example at a rate of 1 g of composition in 100 g of water, and measuring the pH of the solution obtained.

The composition according to the invention comprises less than 5% by weight of water relative to the total weight of the composition. The water content of the composition according to the invention is preferably less than or equal to 4% by weight, preferably less than or equal to 3% by weight, in particular less than or equal to 2% by weight, for example less than or equal to 1% by weight, relative to the total weight of the composition. In specific embodiments of the invention, the composition does not contain water or contains only a trace of water. In alternative embodiments of the invention, the composition comprises 0.5 to 4% by weight of water, in particular 1 to 3% by weight of water, relative to the total weight of the composition.

The composition according to the invention can furthermore comply with one or more of the features described hereinafter, used separately or in each of the technically feasible combinations thereof.

The ingredients contained in the composition according to the invention are preferably natural, thus meeting a very strong demand for formulations containing natural ingredients that has been a feature of the cosmetics market for some years. The composition according to the invention preferably includes at least 80%, preferably at least 90%, and preferentially at least 95%, of natural ingredients according to the calculation approach set out in standard ISO 16128:2017.

In addition to the non-natural ingredients mentioned above, such as the surfactants containing an isethionic acid or isethionate group, and the surfactants containing a sulfosuccinic acid or sulfosuccinate group, and mentioned below, the composition according to the invention is also preferably devoid of paraffin. In addition, the composition according to the invention preferably does not contain any ingredients that irritate the skin, cause dryness, or could result in an adverse or allergic reaction. Thus, the composition according to the invention is preferably devoid of any anionic surfactant comprising at least a sulfate group, i.e. a —O—SO₃H or —O—SO₂—O— group, such as laureth sulfate or lauryl sulfate. Although the sulfated surfactants have particularly good lathering properties, they are indeed hash on the skin.

In addition, still in order to improve its tolerance by users, the composition according to the invention is preferably devoid of any compound having an acrylate function and of silicone.

In preferred embodiments of the invention, the content of said mixture of surfactants is comprised between 15 and 50% by weight, preferably between 20 and 40% by weight, in particular between 20 and 35% by weight, relative to the total weight of the composition. Such value ranges ensure that the composition has good lathering properties and that the lather formed is of good quality, while at the same time being associated with a low manufacturing cost for the composition.

The total content of non-ionic surfactant(s) selected from alkyl glucosides in which the alkyl radical comprises from 8 to 16 carbon atoms is preferably comprised between 1 and 5% by weight, in particular between 1 and 3% by weight, relative to the total weight of the composition.

The total content of anionic surfactant(s) selected from acyl glutamic acids and their cosmetically acceptable salts is preferably comprised between 10 and 35% by weight relative to the total weight of the composition.

The total content of anionic surfactant(s) selected from acyl lactylic acids and their cosmetically acceptable salts is preferably comprised between 2 and 10% by weight relative to the total weight of the composition.

In particular embodiments of the invention, the total content of anionic surfactant(s) selected from acyl glutamic acids and their cosmetically acceptable salts is comprised between 50 and 80% by weight, in particular between 60 and 70% by weight, relative to the total weight of said mixture of surfactants.

The mass ratio of non-ionic surfactant(s) selected from alkyl glucosides in which the alkyl radial comprises from 8 to 16 carbon atoms to anionic surfactant(s) selected from acyl lactylic acids and their cosmetically acceptable salts is preferably comprised between 0.3 and 1, and more preferably between 0.4 and 0.8, in particular comprised between 0.5 and 0.6.

The anionic surfactant(s) selected from acyl glutamic acids and their cosmetically acceptable salts are preferably selected from acyl glutamic acids the acyl group of which comprises a linear or branched, saturated or unsaturated, hydrocarbon chain, with 8 to 30 carbon atoms, preferably 10 to 20 carbon atoms and more preferably 12 to 18 carbon atoms. The acyl group can in particular be chosen from the lauroyl, myristoyl, palmitoyl, stearoyl, olivoyl, cocoyl and oleoyl groups. The cosmetically acceptable salt of the acyl glutamic acid can be chosen from alkali metal salts, alkaline earth metal salts and ammonium salts. It is, for example, chosen from sodium, potassium, lithium or magnesium salts, the sodium and potassium salts being particularly preferred within the scope of the invention. The anionic surfactant(s) selected from acyl glutamic acids and their cosmetically acceptable salts can, for example, be selected from the group consisting of compounds with the INCI names sodium cocoyl glutamate, disodium cocoyl glutamate, sodium myristoyl glutamate, sodium lauroyl glutamate, potassium lauroyl glutamate, potassium cocoyl glutamate and sodium stearoyl glutamate. Several anionic surfactants selected from acyl glutamic acids and their cosmetically acceptable salts can be used in the composition according to the invention, which can, for example, contain any mixture of two or more of the compounds listed above. The composition according to the invention preferably comprises a single anionic surfactant selected from acyl glutamic acids and their cosmetically acceptable salts.

The non-ionic surfactant(s) selected from alkyl glucosides in which the alkyl radical comprises from 8 to 16 carbon atoms are preferably chosen from the compounds with the INCI names octyl glucoside, lauryl glucoside and decyl glucoside. Several non-ionic surfactants selected from alkyl glucosides in which the alkyl radical comprises from 8 to 16 carbon atoms can be used in the composition according to the invention, which can, for example, contain any mixture of two or more of the aforementioned compounds (octyl glucoside, lauryl glucoside and decyl glucoside). The composition according to the invention preferably comprises a single non-ionic surfactant selected from alkyl glucosides in which the alkyl radical comprises from 8 to 16 carbon atoms.

The anionic surfactant(s) selected from acyl lactylic acids and their cosmetically acceptable salts are preferably selected from acyl lactylic acids the acyl group of which comprises a linear or branched, saturated or unsaturated, hydrocarbon chain, with 8 to 30 carbon atoms, preferably 10 to 20 carbon atoms and more preferably 12 to 18 carbon atoms. The acyl group can in particular be chosen from the caproyl, lauroyl, myristoyl, palmitoyl, stearoyl, isostearoyl, olivoyl, cocoyl and oleoyl groups. The cosmetically acceptable salt of the acyl lactylic acid can be chosen from alkali metal salts, alkaline earth metal salts and ammonium salts. It is, for example, chosen from sodium, potassium, lithium or magnesium salts, the sodium and potassium salts being particularly preferred within the scope of the invention. The anionic surfactant(s) selected from acyl lactylic acids and their cosmetically acceptable salts are, for example, chosen from compounds with the INCI names sodium lauroyl lactylate, sodium stearoyl lactylate, sodium caproyl lactylate and sodium isostearoyl lactylate. In preferred embodiments of the invention, the anionic surfactant(s) selected from acyl lactylic acids and their cosmetically acceptable salts necessarily comprise sodium lauroyl lactylate. Several anionic surfactants selected from acyl lactylic acids and their cosmetically acceptable salts can be used in the composition according to the invention, which can, for example, contain a mixture of sodium lauroyl lactylate and sodium stearoyl lactylate. The composition according to the invention preferably comprises a single anionic surfactant selected from acyl lactylic acids and their cosmetically acceptable salts, and preferably it is a lauroyl lactylate salt, for example sodium lauroyl lactylate.

Generally speaking, the composition according to the invention preferably contains as few surfactants as possible. Thus, in particular embodiments of the invention, the composition comprises no more than 6 surfactants, preferably no more than 5 surfactants, more preferably no more than 4 surfactants and preferentially 3 surfactants. As mentioned above, good lathering properties can be achieved with any combination of a single compound from each of the three families of surfactants defined above, advantageously minimising the number of ingredients present in the composition.

The composition according to the invention can contain other anionic surfactants than the ones defined above. It preferably contains no anionic surfactant other than acyl glutamic acids/acyl glutamates and acyl lactylic acids/acyl lactylates. Similarly, the composition according to the invention can contain other non-ionic surfactants than the ones defined above. It preferably contains no non-ionic surfactant other than alkyl glycosides the alkyl radical of which has 8 to 16 carbon atoms. In particular, it preferably does not contain any alkyl glucoside the alkyl radical of which has more than 16 carbon atoms. The presence of such alkyl glucosides in the composition is in particular detrimental to the hardness and stickiness of the composition.

The composition according to the invention can also comprise one or more cationic surfactants, for example when it is intended for the cosmetic treatment of hair, for a conditioning treatment. It is preferably devoid of non-natural cationic surfactant and, preferably, more generally speaking, of cationic surfactant.

The composition according to the invention can also comprise one or more amphoteric surfactants. It is preferably devoid of non-natural amphoteric surfactant, such as betaine surfactants, and, preferably, more generally speaking, of amphoteric surfactant.

In particular embodiments of the invention, the mixture of surfactants of the composition consists of:
one or more, preferably a single, anionic surfactant(s) selected from acyl glutamic acids and their cosmetically acceptable salts,
one or more, preferably a single, non-ionic surfactant(s) selected from alkyl glucosides in which the alkyl radical comprises from 8 to 16 carbon atoms,
and one or more, preferably a single, anionic surfactant(s) selected from acyl lactylic acids and their cosmetically acceptable salts.

The composition according to the invention can also contain one or more emulsifiers, such as glycol palmitate (INCI name). Alternatively, it may not contain any. It is preferably devoid of emulsifier formed by a mixture with the INCI name “arachidyl alcohol, arachidyl/behenyl alcohol, arachidyl glucoside”, such as the mixture marketed under the name Montanov® 202.

More generally, the composition according to the invention is preferably devoid of a mixture or arachidyl alcohol and arachidyl glucoside. Indeed, such a mixture gives the composition a high degree of hardness and stickiness, making it less easy to use.

In particular embodiments of the invention, said at least one cosmetically acceptable excipient is selected from consistency agents, fillers, emollients, conditioning agents, stabilisers, fragrances, dyes, pigments and mixtures thereof.

The composition according to the invention in particular preferably comprises at least one fatty phase consistency agent. The total content of consistency agent(s) in the composition is preferably comprised between 25 and 65% by weight, preferably between 30 and 60% by weight, in particular between 35 and 55% by weight, relative to the total weight of the composition. Such a concentration of consistency agent(s) in the composition advantageously ensures, in combination with the filler(s) which can also be contained therein, as explained below, that the composition has optimum hardness and stickiness properties, in particular a sufficiently low degree of hardness so that the composition does not crumble when it is handled by the user, and a sufficiently high stickiness so that the composition has good cohesion, this without detracting from its practical and pleasant properties.

The composition according to the invention can in particular contain one or more consistency agents chosen from fatty alcohols, preferably C16-C22, fatty acids, in particular C16-C18, and hydrogenated castor oil.

In a particular embodiment of the invention, when the composition comprises, as consistency agents, at least one or more fatty alcohols and one or more fatty acids, the total weight of fatty alcohols is then greater than the total weight of fatty acids.

According to a particular embodiment, the composition according to the invention, when it contains one or more fatty acid(s) as consistency agent(s), therefore also contains at least one fatty alcohol or hydrogenated castor oil.

By way of example, the composition according to the invention can contain one or more of the following ingredients as consistency agent(s): cetostearyl alcohol, for example as sold under the name Lanette® O OR/MB, behenyl alcohol, for example as sold under the name Lanette® 22, a mixture of stearic acid and palmitic acid, for example as sold under the name Stearine TP 1200, hydrogenated castor oil, for example as sold under the name Cutina® HR.

In particular embodiments of the invention, the composition comprises, as cosmetically acceptable excipient, at least one filler, which can either be mineral or organic in origin.

Fillers of mineral origin that can be used in the composition according to the invention include clays, ceramic beads, calcium carbonate, titanium oxides, kaolin or aluminium silicate. Fillers of organic origin include starches, maltodextrin, celluloses such as cellulose beads, microcrystalline cellulose, sodium carbonate, Nylon microspheres, micronised or non-micronised plant-based powders such as fruit powders or bamboo powders, or rice hull powder, and mixtures thereof.

The composition according to the invention preferably contains at least one filler selected from kaolin, maltodextrin, starches, celluloses, calcium carbonate and mixtures thereof.

Starches that can be used in the present invention can be obtained from a plant source such as cereals, tubers, roots, vegetables, and fruit. It can, for example, be corn starch, potato starch, pea starch, rice starch, oat starch, barley starch, tapioca starch, wheat starch, cassava starch or sorghum starch. These starches can be modified or not. A modified starch is a starch which has been modified by methods known to a person skilled in the art, such as esterification, etherification, oxidation, acid hydrolysis, cross-linking or enzyme conversion. It is possible, for example, to use the microcrystalline cellulose as sold under the name Vivapur® CS 9 FM as cellulose. The kaolin sold under the name ImerCare® Opaque by Imerys can, for example, be used.

The total quantity of filler(s) in the composition according to the invention is preferably comprised between 5 and 25% by weight, preferably between 5 and 20% by weight, for example between 15 and 20% by weight, relative to the total weight of the composition.

The composition according to the invention can contain one or more emollients, in particular chosen from vegetable oils, such as sunflower seed oil or sweet almond oil, butters, such as shea butter, for example as sold under the name Lipex SheaLight® or Cetiol® SB 45, lauroyl lysine, for example as sold under the name Amihope® LL, and glycerine.

The emollient content of the composition according to the invention is, for example, comprised between 2 and 20% by weight, in particular between 2 and 15% by weight, preferably between 2 and 10% by weight or between 5 and 15% by weight, relative to the total weight of the composition.

In other particular embodiments of the invention, the emollient content of the composition according to the invention is less than 5 by weight relative to the total weight of the composition.

The composition according to the invention can also contain one or more pH adjusters. It is preferably devoid of pH adjusters, in particular of the NaOH, citric acid, lactic acid, etc. type.

Optionally, the composition according to the invention can also contain:
one or more conditioning agent(s), preferably not belonging to the category of cationic surfactants, in particular chosen from isoamyl laurate, lauroyl lysine, the combination of chitosan and mandelic acid, erythritol, glyceryl oleate, for example as sold under the name DUB® OG, guar, polyglyceryl 4-caprate or any mixture thereof, the total quantity of conditioning agent(s) preferably being comprised between 0.01 and 2% by weight, preferably between 0.05 and 1.5% by weight, relative to the total weight of the composition;
and/or one or more stabiliser(s), such as chelating agents, for example tetrasodium glutamate diacetate, as sold under the name Dissolvine® GL-47-S, vitamin E or tocopheryl acetate, as sold under the name Covi-ox® T-90.

The composition according to the invention can contain one or more cosmetically active agents, in particular topical agents, for example chosen from moisturising agents, antioxidants, vitamins, etc.

The total content of cosmetically active agent(s) in the composition according to the invention can, for example, be comprised between 0.05 and 3% by weight, relative to the total weight of the composition.

In particular embodiments of the invention, the composition comprises one or several cosmetically active agent(s) selected from hyaluronic acid, oat sap, helichrysum extracts, helichrysum essential oils, meadowfoam oil, in particular Limnanthes alba seed oil, oat seedlings, flower acids, such as the product marketed under the name Acides de Fleurs®, Garcinia extracts, milk thistle oil, niacinamide, Edelweiss extracts, such as the product marketed under the name Alpaflor® Edelweiss, cinchona extracts, caffeine, peony extracts, mango butter and citron extracts, such a list in no way limiting the invention.

The composition according to the invention can also contain an exfoliating agent, which will exfoliate the skin (including the scalp) by a mechanical and/or chemical action, exfoliating agents acting both by mechanical action and chemical action being particularly preferred within the scope of the invention. The exfoliating agent can be chosen from agents of mineral, plant, or organic origin. Thus, it is possible to use, for example, polyethylene beads or powder, nylon particles, polyvinyl chloride powder, pumice stone (INCI name: pumice), walnut shells, sawdust, wood flour, cork flour, glass beads, alumina (aluminium oxide) (INCI name: alumina), sugar crystals, beads which melt when applied to the skin, such as spheres based on mannitol and cellulose, agar-based capsules, jojoba ester-based spheres, and mixtures thereof. The exfoliating agent(s) used are preferably not water-soluble.

The composition according to the invention, in its desired form, can be prepared by any conventional method for preparing solid cosmetic compositions. Particularly preferred preparation methods within the scope of the invention include extrusion and hot casting methods.

A method for preparing a composition according to the invention by extrusion comprises preparing a noodle containing the surfactants of the composition and the excipient(s), to which any active agents and fragrance are then added. The mixture is then extruded through various screens to homogenise and refine it. Once the mixture is homogeneous, it produces a soap bar which is cut and shaped to obtain a solid composition in the desired shape.

A method for preparing a composition according to the invention by hot casting comprises mixing the ingredients of the composition and heating the resulting mixture to a temperature of around 75-80° C. The homogenised hot mixture is then poured into moulds of the desired shape, then left to cool.

Both of these preparation methods are easy and quick.

Another aspect of the invention relates to the non-therapeutic cosmetic use of a solid cosmetic composition according to the invention for the washing and/or care (including exfoliation) of the skin, in particular the skin of the body and/or face and/or scalp, and/or hair, of an individual, preferably by topical application. This use preferably comprises consecutive steps of:
wetting the composition with water,
lathering, in particular by rubbing the wet surface of the composition,
applying this lather to the skin and/or hair, which have preferably been wet beforehand,
and rinsing off, preferably with water, if necessary after leaving it on for a while and/or massaging.

These steps can, for example, be carried out once to twice a day, for example in the morning and evening, or as often as required.

The solid composition according to the invention can in particular be used for cleansing and/or caring for sensitive or atopic skin.

In the present description, sensitive skin is understood to mean skin prone to irritation or allergic reactions due to various internal and external non-immunological factors. Atopic skin is understood to mean skin affected by an inflammatory disease characterised by dry, itchy skin, such as eczema or atopic dermatitis.

The invention also relates to a non-therapeutic cosmetic treatment method for hygiene and/or care of the skin and/or hair of an individual, using a solid cosmetic composition according to the invention. This method comprises consecutive steps of:
wetting the composition with water,
lathering,
applying the lather to the skin and/or hair,
and rinsing off, preferably with water.

This method can comply with any feature, or combination of features, described above with reference to the cosmetic use of the composition according to the invention.

The features and advantages of the invention will become more apparent from the exemplary embodiments below, provided merely by way of example and in no way limiting the invention.

### EXAMPLES

### 1/Examples of Compositions

Solid compositions according to the invention, intended for cosmetic treatment, for skin hygiene and care on the body and face, have the characteristics listed in Table 1.

TABLE 1
Examples of compositions according to the invention
for skin hygiene and care on the body and face

| | Composi- | Composi- | Composi- |
|---|---|---|---|
| | tion 1 | tion 2 | tion 9 |
| Ingredient (INCI name) | % w/w | % w/w | % w/w |
| Sodium cocoyl glutamate | 15-20 | 25-30 | 5-10 |
| Sodium myristoyl glutamate | — | — | 5-10 |
| Decyl glucoside | — | 1.5 | 3 |
| Lauryl glucoside | 3 | — | — |
| Sodium lauroyl lactylate | 3-6 | 3-6 | 3-6 |
| Stearic acid/palmitic acid | 7-10 | 7-10 | 7-10 |
| Behenyl alcohol | — | — | Qsp 100 |
| Cetostearyl alcohol | Qsp 100 | Qsp 100 | — |
| Hydrogenated castor oil | 5-10 | 5-10 | 4 |
| Sunflower oil | 11 | 5 | 2 |
| Shea butter | 2 | 1 | 1 |
| Kaolin | — | — | — |
| Corn starch | 18 | 18 | 20 |
| Tetrasodium glutamate | — | — | — |
| diacetate | | | |
| Water | 3 | 1.5 | 3 |

Solid compositions according to the invention, intended for cosmetic treatment, for hair hygiene and care, have the characteristics listed in Table 2.

TABLE 2
Examples of compositions according to
the invention for hair hygiene and care

| | Composi- | Composi- | Composi- |
|---|---|---|---|
| | tion 3 | tion 4 | tion 5 |
| Ingredient (INCI name) | % w/w | % w/w | % w/w |
| Disodium cocoyl | 25-30 | — | — |
| glutamate | | | |
| Sodium cocoyl glutamate | — | 25-30 | 25-30 |
| Decyl glucoside | 3 | — | — |
| Lauryl glucoside | — | 3 | 3 |
| Sodium lauroyl lactylate | 3-6 | 3-6 | 3-6 |
| Stearic acid/palmitic acid | Qsp 100 | 7 | 7 |
| Behenyl alcohol | — | Qsp 100 | — |
| Cetostearyl alcohol | — | — | Qsp 100 |
| Hydrogenated castor oil | — | 7 | 7 |
| Coconut oil | 5 | — | — |
| Sweet almond oil | — | 6 | — |
| Sunflower oil | — | — | 5 |
| Shea butter | — | — | 1 |
| Maltodextrin | 6 | — | — |
| Corn starch | — | 18 | 18 |
| Tocopheryl acetate | 0.05 | — | — |
| Water | 3 | 3 | 3 |

Comparative solid compositions not in accordance with the invention have the characteristics listed in Table 3.

TABLE 3
Examples of compositions not in accordance with the invention

| | Composi- | Composi- | Composi- |
|---|---|---|---|
| | tion 6 | tion 7 | tion 8 |
| Ingredient (INCI name) | % w/w | % w/w | % w/w |
| Sodium cocoyl | 25 | — | 5-10 |
| glutamate | | | |
| Sodium myristoyl | — | — | 5-10 |
| glutamate | | | |
| Sodium cocoyl | — | 20 | — |
| isethionate | | | |
| Decyl glucoside | — | 3 | — |
| Montanov ® 202 | | | 6 |
| (arachidyl glucoside/ | | | |
| behenyl alcohol/ | | | |
| arachidyl alcohol) | | | |
| Sodium lauroyl | 5 | 9 | 5 |
| lactylate | | | |
| Palmitic acid/stearic | 7 | 45.9 | 7 |
| acid | | | |
| Hydrogenated castor | 7 | — | 4 |
| oil | | | |
| Sunflower oil | 5 | 8 | 2 |
| Shea butter | 1 | — | 1 |
| Corn starch | 15 | — | 20 |
| Maltodextrin | — | 11 | — |
| Tetrasodium | — | 0.1 | — |
| glutamate diacetate | | | |
| Water | — | 3 | — |
| Cetostearyl alcohol | Qsp 100 | — | — |
| Behenyl alcohol | — | — | Qsp 100 |

More specifically, Composition 6 is devoid of alkyl glucoside surfactant with a C8-C16 alkyl radical, Composition 7 contains a surfactant containing an isethionate group and does not contain any acyl glutamate surfactant, and Composition 8 does not contain any alkyl glucoside surfactant with a C8-C16 alkyl radical, but contains a mixture of arachidyl glucoside (C20 alkyl) and arachidyl alcohol, in addition to behenyl alcohol.

### 2/Evaluation of the Characteristics of the Compositions

The compositions of Example 1 are assessed on the following criteria:
lathering properties (quality/quantity): lather height, creamy lather, softness of the lather,
good tolerance (physiological pH, hypoallergenic, softer and gentler on the skin),
stability over time,
organoleptic properties (colour, smell),
hardness, as measured by a texture analyser; the composition must have adequate consistency to prevent crumbling but must not be too soft,
stickiness, as measured by a texture analyser; the composition must have an adequate degree of stickiness to prevent crumbling, but not too high in order to be pleasant to use and to be able to be prepared under good conditions, in particular in an extrusion device,
pH: the measurement is carried out using a pH meter after diluting 1 g of the composition in 100 g of water, the aim being to ensure that the pH does correspond to the physiological pH of the skin.

To this end, the following operating protocols are implemented.

A TA.XTplus texture analyser with a penetration and withdrawal speed of 1 mm/s, a trigger force of 5 g, a distance of 8 mm and a Needle P/2N is used to assess hardness and stickiness. The composition is fixed to the table of the texture analyser and the following are measured: the penetration force of the needle (measure of hardness) and the exit force of the needle (assessment of stickiness). The results are expressed in grams.

A 250 ml graduated cylinder with an external diameter of 38.9 cm and a height of 36 cm and a stopper is used to assess the height of the lather. 0.5 g of composition is placed in this cylinder and demineralised water is added to make up 50 ml. The cylinder is capped and then shaken vigorously 10 times. The height of the lather is read and expressed in mm.

The results obtained are shown in Table 4 for the compositions according to the invention Composition 1, Composition 2 and Composition 5, and the comparative compositions not in accordance with the invention Composition 6, Composition 7 and Composition 8.

TABLE 4
Characteristics of the compositions

| Composition | 1 | 2 | 5 | 6 | 7 | 8 |
|---|---|---|---|---|---|---|
| pH | 5.35 | 5.35 | 5.38 | 5.41 | 4.94 | — |
| Height of the | 42 | 52 | 31 | 22 | 26 | — |
| lather (mm) | | | | | | |
| Hardness (g) | 942 | 1378 | 1236 | 2524 | 1160 | 5995 |
| Stickiness (g) | −397 | −406 | −338 | −988 | −319 | −1573 |

It can be seen that the three compositions according to the invention have a greater lather height than that of the compositions not in accordance with the invention, and in particular of Composition 7 which contains an isethionate surfactant. The lathering properties of Compositions 1 and 5, whose total surfactant content is 23 or 24% w/w, are greater than those of the comparative Compositions 6 and 7, whose surfactant content is much higher (30 or 32% w/w). The surfactant content of Composition 2 according to the invention (31.5% w/w) is similar to that of the latter, and it can be seen that its lathering properties are far superior.

In addition, Composition 6, which does not contain any C8-C16 alkyl glucoside surfactant, is both much stickier and harder than the compositions according to the invention. The hardness and stickiness are even greater still for Composition 8, in which the C8-C16 alkyl glucoside surfactant is replaced by arachidyl glucoside.

Furthermore, the lather height was assessed by the same method for Composition 9 according to the invention. A lather height of 45 mm was obtained, just as satisfactory as the lather heights of Composition 1 and Composition 2.

### 3/Comparative Study of the Sensory Profiles

Composition 10 according to the invention, and Composition 11 not in accordance with the invention, made up of non-natural ingredients such as disodium lauryl sulfosuccinate, sodium cocoyl isethionate and paraffin, having the compositions shown in Table 5 below, were analysed for their sensory profiles.

TABLE 5
Examples of a composition according to the invention
for skin hygiene and care on the body and face,
and of a comparative composition.

| | Composition | |
|---|---|---|
| | Composition 10 | Composition 11 |
| Ingredient (INCI name) | % w/w | % w/w |
| Sodium cocoyl glutamate | 10-15 | — |
| Decyl glucoside | 3 | — |
| Sodium lauroyl lactylate | 3-6 | — |
| Disodium lauryl | — | 25-28 |
| sulfosuccinate | | |
| Sodium cocoyl isethionate | — | 18-20 |
| Avena sativa extract/juice | 1 | — |
| Sodium hyaluronate | 0.1 | — |
| Sweet almond oil | — | 1.5 |
| Palm oil | — | 1 |
| Stearic acid/palmitic acid | 6-10 | 11-12 |
| Behenyl alcohol | Qsp 100 | — |
| Cetostearyl alcohol | — | 2-3 |
| Hydrogenated castor oil | 3-6 | — |
| Sunflower oil | 3 | — |
| Shea butter | 1 | — |
| Paraffin | — | 2-3 |
| Maltodextrin | — | 18-19 |
| Corn starch | 18-20 | — |
| Tetrasodium glutamate | — | — |
| diacetate | | |
| Water | 3 | 7 |

The sensory profile was assessed monadically by 15 trained panellists, after 7 applications of the composition to each wet arm using descriptors specific to the 3 assessment phases:
at application (lather generation, quality and quantity of lather, behaviour of the product between the fingers: oily/tacky sensation),
when rinsing off (easy to rinse off),
and 15 minutes after rinsing off, on dry skin (powdery feel, skin elasticity, tightness, shine, residual film on the skin, oily/resistant/sticky feel).

The conclusions of the test were as follows: the 2 compositions assessed have very similar sensory characteristics. The composition according to the invention Composition 10 was even found to be slightly less oily to the touch and easier to rinse off.

This assessment demonstrates that the composition according to the invention, which is more natural, achieves results on the skin that are at least comparable to the control composition comprising almost 50% of the isethionate/sulfosuccinate combination.

1.-19. (canceled)

20. A solid cosmetic composition, containing:
a mixture of surfactants comprising at least one anionic surfactant selected in the group consisting of acyl glutamic acids and their cosmetically acceptable salts, at least one non-ionic surfactant selected in the group consisting of alkyl glucosides in which the alkyl radical comprises from 8 to 16 carbon atoms, and at least one anionic surfactant selected in the group consisting of acyl lactylic acids and their cosmetically acceptable salts,
and at least one cosmetically acceptable excipient,
said composition comprising less than 5% by weight of water relative to the total weight of the composition, and
said composition being devoid of surfactant containing an isethionic acid or isethionate group and of surfactant containing a sulfosuccinic acid or sulfosuccinate group.

21. The composition according to claim 20, wherein the content of said mixture of surfactants is comprised between 15 and 50% by weight relative to the total weight of the composition.

22. The composition according to claim 20, wherein the total content of non-ionic surfactant(s) selected in the group consisting of alkyl glucosides in which the alkyl radical comprises from 8 to 16 carbon atoms is comprised between 1 and 5% by weight relative to the total weight of the composition.

23. The composition according to claim 20, which is devoid of surfactant comprising a sulfate group.

24. The composition according to claim 20, wherein the total content of anionic surfactant(s) selected in the group consisting of acyl glutamic acids and their cosmetically acceptable salts is comprised between 50 and 80% by weight relative to the total weight of said mixture of surfactants.

25. The composition according to claim 20, wherein said at least one non-ionic surfactant is octyl glucoside, lauryl glucoside, decyl glucoside or one the mixtures thereof.

26. The composition according to claim 20, wherein said at least one anionic surfactant selected in the group consisting of acyl glutamic acids and their cosmetically acceptable salts is selected in the group consisting of sodium cocoyl glutamate, disodium cocoyl glutamate, sodium myristoyl glutamate, sodium lauroyl glutamate, potassium lauroyl glutamate, potassium cocoyl glutamate, sodium stearoyl glutamate and any mixture thereof.

27. The composition according to claim 20, wherein said at least one anionic surfactant selected in the group consisting of acyl lactylic acids and their cosmetically acceptable salts is sodium lauroyl lactylate, sodium stearoyl lactylate or a mixture thereof.

28. The composition according to claim 20, which is devoid of amphoteric surfactant.

29. The composition according to claim 20, wherein said at least one cosmetically acceptable excipient is chosen in the group consisting of consistency agents, fillers, emollients, conditioning agents, stabilisers, fragrances and mixtures thereof.

30. The composition according to claim 29, comprising at least one filler chosen in the group consisting of kaolin, maltodextrin, starches, celluloses, calcium carbonate and mixtures thereof.

31. The composition according to claim 29, comprising a total quantity of filler(s) comprised between 5 and 25% by weight relative to the total weight of the composition.

32. The composition according to claim 29, containing a total quantity of consistency agent(s) comprised between 25 and 65% by weight relative to the total weight of the composition.

33. The composition according to claim 20, also containing an exfoliating agent.

34. The composition according to claim 20, comprising at least one cosmetically active agent selected in the group consisting of hyaluronic acid, oat sap, helichrysum extracts, helichrysum essential oils, meadowfoam oil, oat seedlings, flower acids, Garcinia extracts, milk thistle oil, niacinamide, Edelweiss extracts, cinchona extracts, caffeine, peony extracts, mango butter and citron extracts.

35. The composition according to claim 20, packaged in the form of a rectangular bar, round bar, stick or ball.

36. Non-therapeutic cosmetic treatment method for hygiene and/or care of the skin and/or hair, by means of a composition according to claim 20, comprising the consecutive steps of:
wetting said composition with water,
lathering,
applying said lather to the skin and/or hair,
and rinsing off.

37. The method of claim 36, for cleansing and/or caring for sensitive skin.
